(19) United States
(12) Patent Application Publication
(10) Pub. No.: US 2011/0039927 A1
(43) Pub. Date: Feb. 17, 2011

(54) COMPOSITIONS COMPRISING AND PROCESSES FOR PRODUCING INORGANIC SALTS OF HOP ACIDS

(75) Inventors: Kevin K. Madsen, Yakima, WA (US); Mark M. Bossert, Yakima, WA (US); Mitsunori Ono, Lexington, MA (US)

Correspondence Address: EDWARDS ANGELL PALMER & DODGE LLP, P.O. BOX 55874, BOSTON, MA, 02205, US

(73) Assignee: John I. Haas, Washington, DC (US)

(21) Appl. No.: 11/922,731

(22) PCT Filed: Jun. 21, 2006
(86) PCT No.: PCT/US2006/024045

§ 371 Date: Oct. 19, 2009

Related U.S. Application Data
(60) Provisional application No. 60/692,746, filed on Jun. 21, 2005.
(60) Provisional application No. 60/692,910, filed on Jun. 21, 2005.
(60) Provisional application No. 60/749,966, filed on Dec. 12, 2005.

(30) Foreign Application Priority Data
Apr. 24, 2006 (US) 11/302308

(51) Int. Cl. A61K 31/315; C07C 61/08; C07F 3/06; C07F 15/00; A61K 31/295; A61K 31/19; A61P 17/10; A61P 37/00

(52) U.S. Cl. 514/494; 562/400; 556/118; 556/138; 514/502; 514/557

(57) ABSTRACT

The invention relates to compositions and processes for producing novel hop acid formulations having improved bioavailability, and the use of such compositions as anti-inflammatory agents, dietary supplements, and pharmaceuticals.

### CROSS-REFERENCE TO RELATED APPLICATIONS

This application claims the benefit of the following U.S. Provisional Applications: Ser. No. 60/692,746, filed Jun. 21, 2005; Ser. No. 60/692,910 filed Jun. 21, 2005, and Ser. No. 60/749,966, filed Dec. 12, 2005; this application is a continuation-in-part of U.S. Utility application Ser. No. 11/302,308, which in turn claims the benefit of each of the aforementioned U.S. Provisional Applications. The entire contents of each of these applications is incorporated herein by reference.

### BACKGROUND

Hops have been used for centuries to flavor beer and are considered, along with water, yeast and malt, to be an essential ingredient of beer. A goal of present brewing technology is to make reproducible brews. Compositions and methods that improve the reproducibility of hop flavors are useful for controlling and standardizing the flavoring of beer and ale.

Recent reports have proposed that acids derived from hops are useful as anti-inflammatory agents and dietary supplements. Such acids are likely to have other pharmaceutical applications as well. Because hop acids occur in the form of viscous oils or resins, the active ingredients are poorly absorbed when ingested or topically applied, and are inconvenient to handle. In addition, methods of producing them are time-intensive, which increases production costs. A need exists for hop acid formulations having improved bioavailability and convenience of handling.

Previous methods for producing solid salts of hop acids, such as those described in U.S. Pat. No. 5,624,701, require a four step process that includes heating an aqueous alkaline solution of a hop acid with an aqueous salt solution to produce a solid salt of a hop acid. This heating step accelerates the degradation of alpha acids during salt formation and is undesirable. In addition, this process uses hop acids at concentrations between 4% and 7% during the magnesium salt formation reaction. These low concentrations of hop acids increase the time required for the reaction, which increases costs. Improved methods of converting hop acids to solid salts of hop acids are required.

### SUMMARY

The present invention provides hop acid compositions and methods for producing these compositions. Advantageously, the methods of the invention have greatly improved efficiency for making the inorganic salts of isoalpha acids and reduced isoalpha acids, including rhoisoalpha acids, tetrahydroisoalpha acids, and hexahydro-isoalpha acids; as well as for the production of the inorganic salts of the beta acids and derivatives thereof. In particular, these beta acids include hexahydrobeta acids and tetrahydrobeta acids. In one embodiment, the hop acid is a beta acid selected from the group consisting of lupulone, colupulone, adlupulone and derivatives thereof. In another embodiment, the beta acids are hexahydrobeta acids or tetrahydrobeta acids. These improved methods are 5-10 times faster than previously described methods, which advantageously reduces labor, energy, and other production costs. Another advantage of the present methods is that they do not require a heating step, but are carried out at room temperature. This reduces the degradation of hop acids during salt formation.

Compositions of the invention comprise novel hop acid formulations having improved bioavailability. The invention provides for the use of such compositions as anti-inflammatory agents, dietary supplements, and pharmaceuticals. Such compositions are useful for treating disease or disease symptoms, including those associated with inappropriate inflammation.

In one aspect, the invention features a process for the production of an inorganic salt (e.g., lithium, sodium, potassium, silver, copper, magnesium, calcium, barium, chromium, manganese, iron, silver, cobalt, nickel, copper, zinc, and cadmium) of an alpha acid, reduced isoalpha acid (e.g., isoalpha acid, rhoisoalpha acid, tetrahydroisoalpha acid, or hexahydroisoalphaacid) or a beta acid (e.g., hexahydrobeta acid, tetrahydro beta acid, lupulone, colupulone, adlupulone or derivatives thereof). The method involves (a) providing an aqueous composition or solution containing 10-50% of an isoalpha acid or reduced isoalpha acid, wherein the solution is at room temperature; (b) adding an inorganic salt (e.g., lithium, sodium, potassium, silver, copper, magnesium, calcium, barium, chromium, manganese, iron, silver, cobalt, nickel, copper, zinc, and cadmium) to the aqueous solution with agitation to form a slurry, where the slurry is at room temperature (e.g., between 15 and 25° C.); (c) mixing until the slurry is homogeneous; and (d) drying (e.g., spray drying, vacuum drying, drum drying, pan drying, window drying, and/or freeze drying) the slurry to obtain an inorganic salt of a hop acid. In one embodiment, the method further comprises the step of filtering the slurry of step (c) prior to step (d). In another embodiment, the aqueous composition or solution is an aqueous alkaline solution.

In another aspect, the invention features a process for the production of a magnesium salt (e.g., magnesium sulfate) of a reduced isoalpha acid, including rhoisoalpha acids, tetrahydroisoalpha acids or hexahydroisoalpha acids. The method involves (a) providing an aqueous solution containing 10-50% of a reduced isoalpha acid, where the solution is at room temperature; (b) adding an inorganic magnesium salt to the aqueous solution with agitation to form a slurry, wherein the slurry is at room temperature; (c) mixing until the slurry is homogeneous; and (d) drying the slurry to obtain a magnesium salt of a reduced isoalpha acid. In one embodiment, the method further comprises the step of filtering the slurry of step (c) prior to step (d). In another embodiment, the aqueous solution is an aqueous alkaline solution.

In yet another aspect, the invention features a process for the production of a calcium salt of a reduced isoalpha acid, including rhoisoalpha acids, tetrahydroisoalpha acids or hexahydroisoalpha acids. The method involves (a) providing an aqueous solution containing 10-50% of a reduced isoalpha acid, where the solution is at room temperature; (b) adding an inorganic calcium salt (e.g., calcium carbonate, calcium chloride, or calcium hydroxide) to the aqueous solution with agitation to form a slurry, where the slurry is at room temperature; (c) mixing until the slurry is homogeneous; and (d) drying the slurry to obtain a calcium salt of a reduced isoalpha acid. In one embodiment, the method further comprises the step of filtering the slurry of step (c) prior to step (d). In another embodiment, the aqueous solution is an aqueous alkaline solution.

In various embodiments of the above aspects, the concentration of alpha acid or reduced isoalpha acid present in the aqueous solution is between 10% and 50% (e.g., 10%, 15%, 20%, 25%, 30%, 35%, 40%, 45%, or 50%). In other embodiments of the above aspects, the magnesium/alpha acid or reduced isoalpha acid or calcium/alpha acid or reduced isoalpha acid molar ratio is in a range between 0.3 and 0.8 (e.g., 0.3, 0.4, 0.5, 0.6, 0.7, and 0.8). In other embodiments of any of the above aspects, room temperature is between 15 and 25° C. (e.g., 15, 16, 17, 18, 19, 20, 21, 22, 23, 24, or 25° C.).

In another aspect, the invention features a reduced isoalpha acid, including rhoisoalpha acids, tetrahydroisoalpha acids or hexahydroisoalpha acids made by the process of any of the above aspects.

In another aspect, the invention provides a process for the production of a magnesium salt of a beta acid and/or a derivative of a beta acid, including tetrahydrobeta acids and/or hexahydrobeta acids. The method involves providing an aqueous alkaline solution containing 10-50% of a beta acid, where the solution is at room temperature; adding an inorganic magnesium salt to the aqueous alkaline solution with agitation to form a slurry, where the slurry is at room temperature; mixing until the slurry is homogeneous; and drying the slurry to obtain a magnesium salt of a beta acid. In one embodiment, the magnesium salt is magnesium sulfate. In another embodiment, the method further comprises the step of filtering the slurry of step (c) prior to step (d).

In another aspect, the invention provides a process for the production of a calcium salt of beta acid. The method involves providing an aqueous alkaline solution containing 10-50% of a beta acid, where the solution is at room temperature (e.g., between 15° C. and 25° C., including 17, 18, 19, 20, 21, 22, 23, 24, or 25° C.) adding an inorganic calcium salt to the aqueous alkaline solution with agitation to form a slurry, where the slurry is at room temperature; mixing until the slurry is homogeneous; and drying the slurry to obtain a calcium salt of a beta acid. In one embodiment, the method further comprises the step of filtering the slurry of step (c) prior to step (d). In another embodiment, the calcium salt is at least one of calcium carbonate, calcium chloride, or calcium hydroxide. In another embodiment, the concentration of a beta acid present in the aqueous alkaline solution is between 10% and 45% (e.g., any integer between 10 and 50, wherein the bottom of the range is between 10 and 49, and the top of the range is an integer between 11 and 50; exemplary integers include 10, 15, 20, 25, 30, 35, 40, or 45%); is between 15% and 45%; or is 20%. In yet another embodiment, the inorganic salt/beta molar ratio (e.g., magnesium/beta acid or calcium/beta acid molar ratio) is in a range between 0.3 and 0.8 (e.g., 0.3, 0.4, 0.5, 0.6, 0.7, or 0.8). In yet another embodiment, the drying is accomplished by a method selected from the group consisting of spray drying, vacuum drying, drum drying, pan drying, window drying and freeze drying, or any combination thereof. In still other embodiments, the beta acid is selected from the group consisting of tetrahydrobeta acids, and hexahydrobeta acids.

In yet another aspect, the invention provides a beta acid made by the process of any previous aspect.

In various embodiments of any of the above aspects, the aqueous solution is an aqueous alkaline solution. By “aqueous alkaline solution” is meant any solution having a basic pH, i.e., a pH greater than neutral. In general, a neutral pH is about 7. Accordingly, an aqueous alkaline solution has a pH greater than 7, for example, a pH between 7.4 and 12 (e.g., 7.4, 7.6, 7.8, 8, 9, 10, 11, or 12), inclusive.

Hop acid derivatives are compounds that are chemically derived (either through natural biosynthetic procesess (e.g., living organism metabolism (e.g., mammal, plant, bacetria)) or synthetic processes using human intervention (e.g., chemical synthesis)) from hop acids. Alpha acid derivatives (e.g., isoalpha acids, rhoisoalpha acids, tetrahydroisoalpha acids, and hexahydroisoalpha acids) are compounds derived from hop alpha acids.

The invention further provides compositions containing inorganic salts of beta acids, such as lupulone, colupulone, adlupulone and their derivatives. Beta acid derivatives are compounds derived from hop beta acids. In particular embodiments, beta acids include hexahydrobeta acids and tetrahydrobeta acids.

In another aspect, the present invention provides compositions containing a composition comprising any one or more of Formulas A, B, C and D:

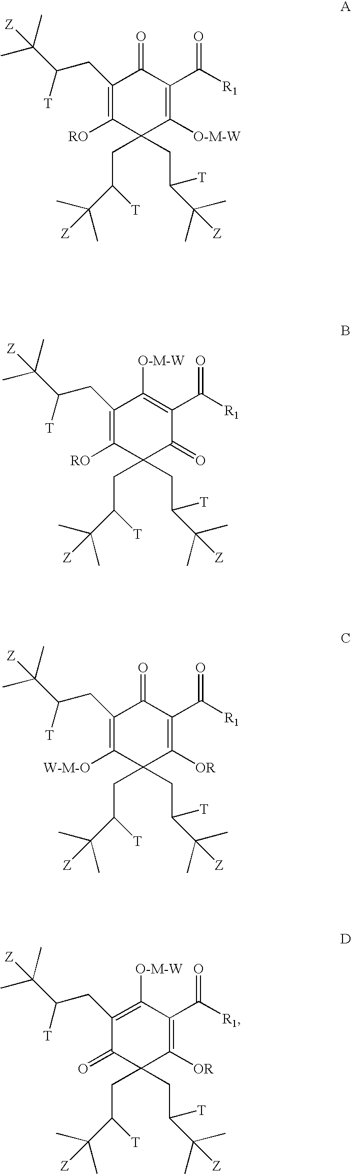

where R1 is alkyl;
where Z and T are independently selected from H and Pi-orbital with the proviso that if one of T or Z is a Pi orbital, then the adjacent T or Z is also a Pi orbital, thereby forming a double bond;
where M is a monovalent or divalent cation;
where W is Cl, OH, SO4⁻, Br, I, or a compound of Formula E, F, G or H:

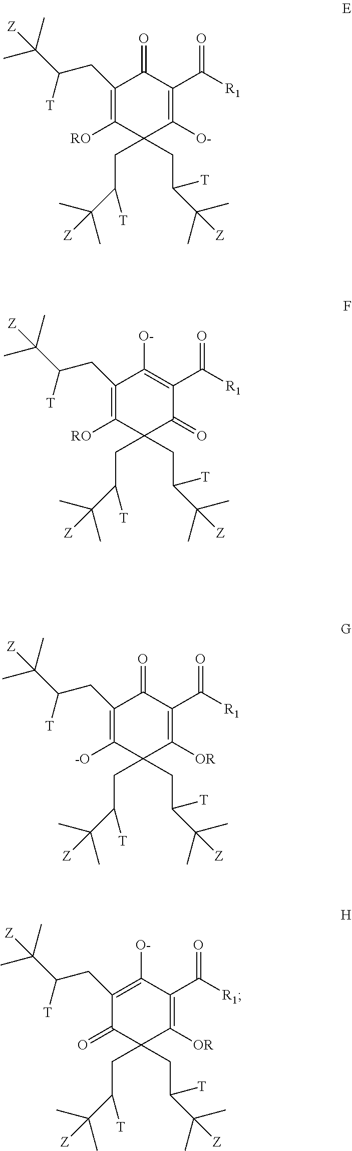

and where R is H, Na, K, Li or M-W. Advantageously, the magnesium salts of a beta acid are much less hygroscopic than the beta acids themselves.

In one aspect, the present invention provides aqueous compositions containing between 1% and 95% inclusive inorganic salts of beta acids, hexahydrobeta acids and tetrahydrobeta acids. Such aqueous formulations have improved bioavailability and are suitable for oral or topical administration to a subject. In addition, the production of such formulations is more efficient than prior art methods, and the aqueous formulations are more convenient to handle than prior art formulations.

In one aspect, the invention features a composition containing an aqueous composition having 1-95% (e.g., 1%, 3%, 5%, 10%, 20%, 30%, 40%, 50%, 60%, 70%, 80%, 90%, or 95%) inclusive of a combination of Formulas A, B, C and D:

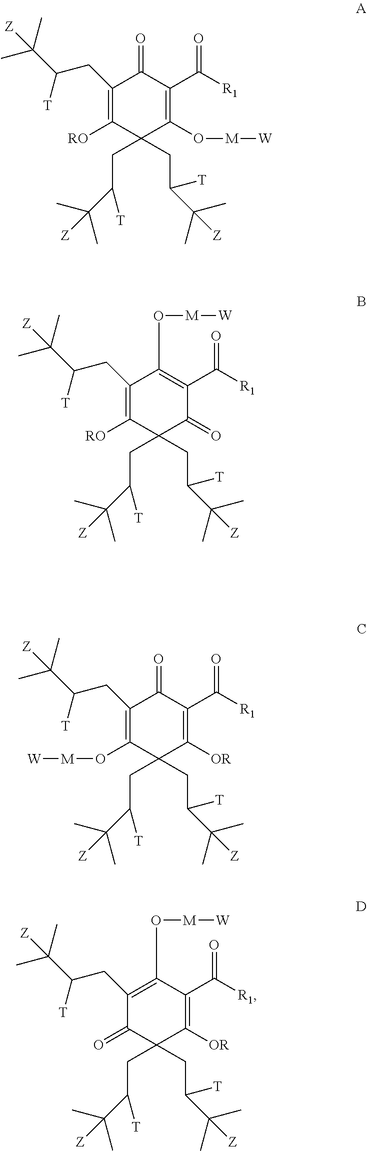

where R1 is alkyl;
where Z and T are independently selected from H and Pi-orbital with the proviso that if one of T or Z is a Pi orbital, then the adjacent Tor Z is also a Pi orbital, thereby forming a double bond;
where M is magnesium or calcium;
where W is Cl, OH, SO4⁻, Br, I, or a compound of Formula E, F, G or H:

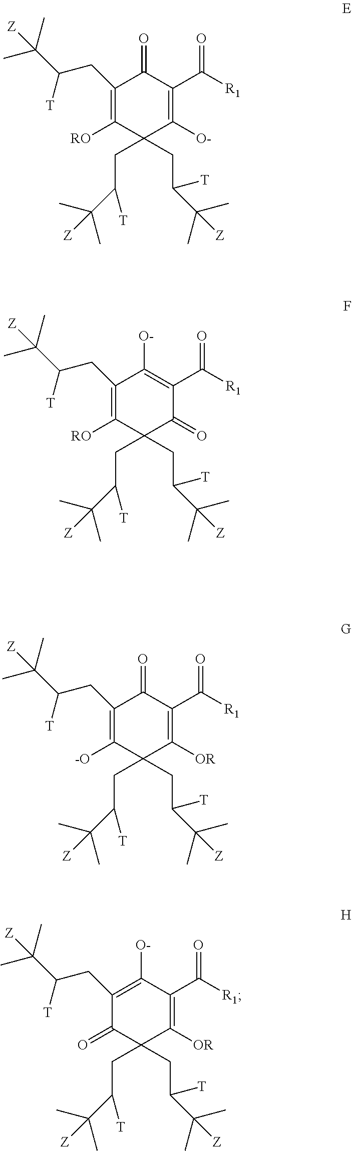

and where R is H, Na, K, Li or M-W.

In one embodiment, the aqueous composition contains at least one hop acid selected from the group consisting of beta acids, hexahydrobeta acids and tetrahydrobeta acids, or a natural or synthetic derivative thereof. In another embodiment, the pH of the aqueous composition is between 7.0 and 10.0 (e.g., 7.0, 7.2, 7.4, 7.6, 7.8, 8.0, 8.2, 8.4, 8.6, 8.8, 9.0, 9.2, 9.4, 9.5), inclusive. In yet other embodiments, the pH is between 7.0 and 10.0, 7.0 and 9.5, 7.0 and 8.0, or is between 7.2 and 7.4. In still other embodiments, the water content of the aqueous composition is between 1-95% (e.g., 1%, 3%, 5%, 10%, 20%, 25%, 30%, 40%, 50%, 60%, 70%, 80%, 90% 95%), inclusive. In various embodiments, the lower limit of the range is any number between 1% and 94% and the upper limit of the range is any number between 4% and 95%. In other embodiments, the water content of the aqueous composition is between 1-10%, between 1-20%, between 20-90%, between 15-25%, between 30-50%, between 55-75%, or between 80-95%.

In another aspect, the invention features a composition containing at least one of Formulas A, B, C or D:

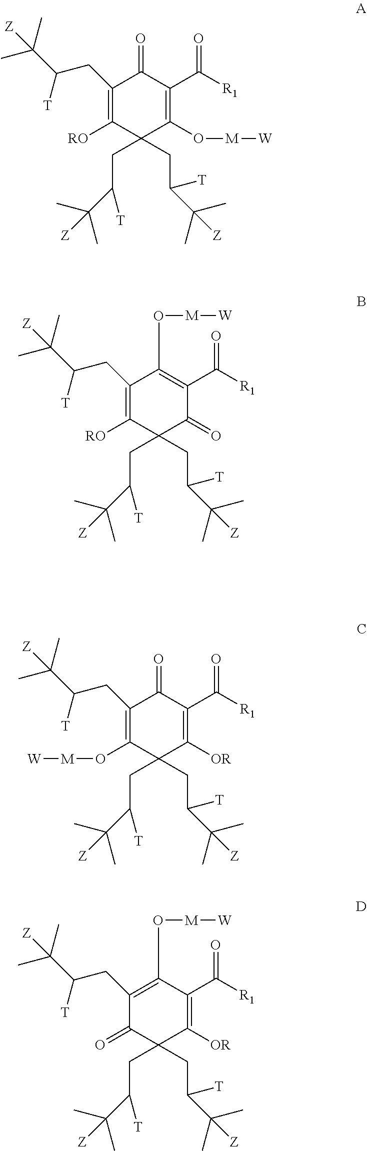

where R1 is alkyl;
where Z and T are independently selected from H and Pi-orbital, with the proviso that if one of T or Z is a Pi orbital, then the adjacent T or Z is also a Pi orbital, thereby forming a double bond;
where M is magnesium or calcium;
where W is Cl, OH, SO4-, Br, 1, or a compound of Formula E, F, G or H:

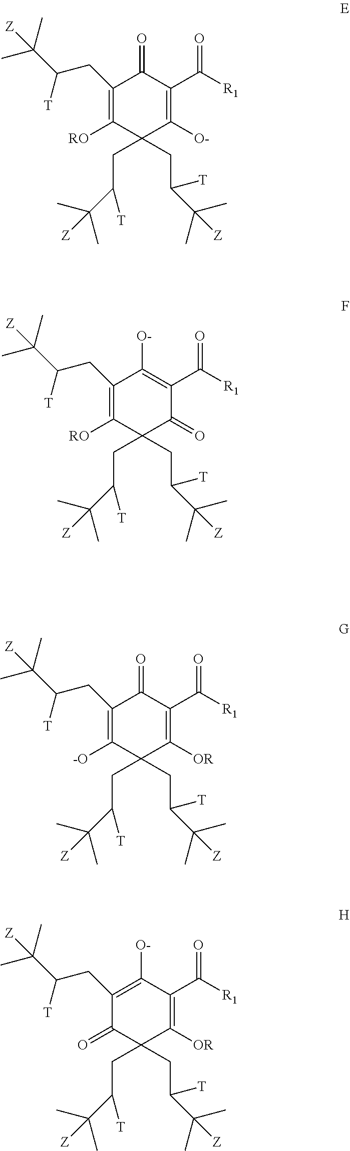

and where R is H, Na, K, Li or M-W.

Exemplary rhoisoalpha acids that may be made by the process of the invention include, but are not limited to, any one or more of the following formulas:

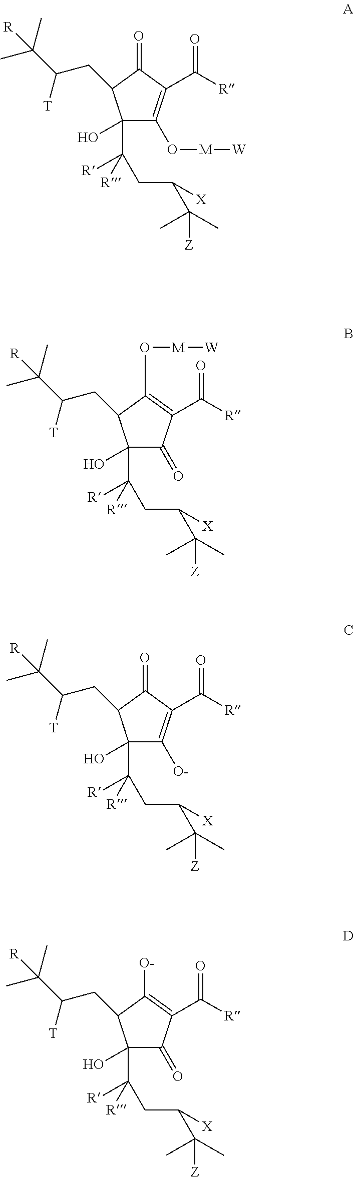

where R′ is selected from the group consisting of hydroxyl, OR and OCOR, R is independently alkyl, and R H; or R′″ and R′″ taken together are ═O; and where R″ is alkyl;
where R, T, X and Z are independently selected from the group consisting of H, F, Cl, Br, I and Pi-orbital, with the proviso that if one of R, T, X, or Z is a Pi orbital, then the adjacent R, T, X, or Z is also a Pi orbital, thereby forming a double bond;
where M is magnesium or calcium;

where W is Cl, OH, SO4-, Br, I, Formula C or Formula D.

In one particular embodiment, the reduced isoalpha acid (e.g., rhoisoalpha acid) is a hexahydroisoalpha acid having the following formula:

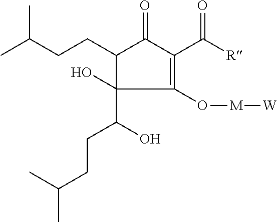

where R″ is alkyl;
where M is lithium, sodium, potassium, silver, copper, magnesium, calcium, barium, chromium, manganese, iron, silver, cobalt, nickel, copper, zinc, or cadmium; and where W is absent or is Cl, OH, SO4-, Br, or I.

In various embodiments of any of the compositions featured herein, M is a monovalent or divalent cation selected from the group consisting of lithium, sodium, potassium, silver, copper, magnesium, calcium, barium, chromium, manganese, iron, silver, cobalt, nickel, copper, zinc, and cadmium. In one embodiment, M is sodium, potassium, calcium, iron, or zinc. The number of occurrences of W are selected to provide charge balance for the metal ion M, such that the entire complex is charge neutral. Thus, W can represent from 0-2 occurrences of a selected substituent. For example, where M is Na, W is absent; where M is calcium, then W will represent a single occurrence of a substituent that provides charge balance. In various embodiments, W is acetate, adipate, alginate, aspartate, benzoate, benzenesulfonate, bisulfate, bromide, butyrate, citrate, camphorate, camphorsulfonate, chloride, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, hydroxyl, iodide, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. While the compounds of the invention are depicted herein in a particular form, the invention is not so limited. Other tautomeric forms (e.g., keno/enol tautomers) are within the scope of the invention.

In still other aspects the invention provides a composition (e.g., an aqueous composition) containing an inorganic salt of a beta acid, where the beta acid is any one or more of lupulone, colupulone, adlupulone, hexahydrobeta acids and tetrahydrobeta acids. In various embodiments the salt contains a monovalent or divalent cation (e.g., a metal), such as lithium, sodium, potassium, silver, copper, magnesium, calcium, barium, chromium, manganese, iron, cobalt, nickel, copper, zinc, or cadmium.

In still other aspects, the invention provides an inorganic salt of a reduced isoalpha acid, wherein the isoalpha acid is selected from the group consisting of rhoisoalpha acids, tetrahydroisoalpha acids, and hexahydroisoalpha acids. In various embodiments, the salt contains a monovalent or divalent cation (e.g., a metal), such as lithium, sodium, potassium, silver, copper, magnesium, calcium, barium, chromium, manganese, iron, cobalt, nickel, copper, zinc, cadmium.

In various embodiments of any of the previous aspects, the invention features a composition containing between 1-95% (e.g., 1%, 5%, 10%, 20%, 30%, 40%, 50%, 60%, 70%, 80%, 90%, or 95%), inclusive of a combination of any one or more of (e.g., 2, 3, or 4) Formulas A, B, C or D. In one embodiment, the invention features a composition containing between 3-95% of a combination of Formulas A, B, C, or D. In various other embodiments, the lower limit of the range is any number between 1% and 94% and the upper limit of the range is any number between 4% and 95%. In yet other embodiments, the composition contains between 3% and 10%, between 10% and 25%, between 30% and 50%, between 60% and 75%, or between 80% and 95% of Formulas A and B. In yet another embodiment, the composition contains at least one hop acid selected from the group consisting of beta acids, hexahydrobeta acids and tetrahydrobeta acids.

In various embodiments of any of the above aspects, W is magnesium or calcium. In other embodiments of any of the above aspects, the composition contains magnesium salts, calcium salts, potassium salts, lithium salts, iron salts, zinc salts, calcium and magnesium salts, or combinations of these salts or any one or more of (e.g., 2, 3, or 4) Formulas A, B, C or D. In other embodiments of the above aspects, the pH of the aqueous composition is between 7.0 and 9.5 (e.g., 7.0, 7.2, 7.4, 7.6, 7.8, 8.0, 8.2, 8.4, 8.6, 8.8, 9.0, 9.2, 9.4, 9.5). In yet other embodiments of any of the above aspects, the inorganic salt/beta acid molar ratio (e.g., magnesium/beta acid or calcium/beta acid molar ratio) is in a range between 0.3 and 0.8 (e.g., 0.3, 0.4, 0.5, 0.6, 0.7, 0.8). In one embodiment of any of the above aspects, the magnesium or calcium/beta acid molar ratio is in a range between 0.4 and 0.6. In related embodiments, the lower limit of the range is any number between 0.3 and 0.79 and the upper limit of the range is any number between 0.35 and 0.8. In yet other embodiments of any of the above aspects, the composition further contains at least one, two, or three of curcuminoids, stilbenoids, or flavonoids.

In another aspect, the invention features an anti-inflammatory, an anti-microbial, or an anti-bacterial composition containing the composition of any of the previous aspects in a pharmaceutically acceptable carrier. In one embodiment, the composition further contains at least one additional therapeutic agent. In other embodiments, the additional therapeutic agent is selected from the group consisting of immunosuppressors (e.g., azathioprine, 6-mercaptopurine, cyclosporine A, tacrolimus, cyclophosphamide, and methotrexate, anti-inflammatory agents, and steroids), anti-inflammatory agents (e.g., sulfasalazine, olsalazine, mesalmine, ibuprofen, ketoprofen, piroxicam, naproxen sodium, sulindac, aspirin, choline subsalicylate, diflunisal, oxaproxin, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate), and steroids (e.g., cortisone, budesonide, and prednisone).

In yet another aspect, the invention features a nutraceutical composition containing a composition of any one of the previous aspects in a nutraceutically acceptable carrier. In one embodiment, the nutraceutical composition further contains at least one edible plant extract (e.g., turmeric). In other embodiments, the composition contains curcuminoids, stilbenoids, or flavonoids. In yet another embodiment, the composition contains curcumin or resveratrol.

In one aspect, the invention features a non-alcoholic food product (e.g., milk, tea, soft drink, juice, coffee, seasoning, cereal, water, yogurt, cookies, chewing gum, chocolate, or soup) containing the composition of any previous aspect. In other embodiments, the composition contains curcuminoids, stilbenoids, or flavonoids. In yet another embodiment, the composition contains curcumin or resveratrol.

In yet another aspect, the invention features a dietary supplement containing the composition of any previous aspect. In one embodiment, the composition further contains curcuminoids, stilbenoids, or flavonoids. In yet another embodiment, the composition contains curcumin or resveratrol.

In yet another aspect, the invention features a method of ameliorating an inflammatory disease or disease symptom in a subject (e.g., a human patient) in need of such treatment containing administering to the subject an effective amount of a composition of any previous aspect. In one embodiment, the disease or disease symptom is any one or more of an autoimmune disorder, asthma, atherosclerosis, chronic inflammatory bowel disease, colitis, coronary artery disease, eczema, inflammatory dermatoses, juvenile and rheumatoid arthritis, pruritis/inflanunation, osteoarthritis, psoriasis, systemic lupus erythematosus, type 2 diabetes, and ulcerative colitis.

In another aspect, the invention features a method of treating a pathogen infection or a disease symptom thereof. In one embodiment, the pathogen infection is a bacterial infection (e.g., an infection with Helicobacter, such as H. pylori, Mycobacterium, such as M. tuberculosis, or Propionibacterium acnes)

In another aspect, the invention provides a method of treating a bacterial infection using a composition of any of the above aspects. In one embodiment, the bacterial infection results in a dermatological disease, such as acne vulgaris or acne.

Exemplary bacteria susceptible to treatment with a composition of the invention include any one or more of Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

In yet another aspect, the invention features a method of reducing an inflammatory response in a subject containing administering to the subject an effective amount of a composition of any one of the above aspects.

One aspect is a composition having at least one compound selected from hop acids, such as hop beta acids, hexahydrobeta acids and tetrahydrobeta acids, their derivatives, and their use to provide a method of treating inappropriate inflammation and related diseases, by administering to a subject the aforementioned compositions.

One embodiment of the present invention is an anti-inflammatory composition comprising a component having at least one compound that is a hop acid (e.g., beta acids, hexahydrobeta acids and tetrahydrobeta acids). In other aspects, the combination of beta acids, hexahydrobeta acids and tetrahydrobeta acids is about 85%, about 90%, about 95%, or about 98% of the hop acids in the entire composition. In other aspects, each of beta acids, hexahydrobeta acids and tetrahydrobeta acids is about 65%, about 70%, about 75%, about 80%, about 85%, about 90% or about 95% of the beta acid(s) in the entire composition.

Another embodiment is an anti-inflammatory composition comprising a component having at least one compound that is a beta acid or beta acid derivative. Particularly preferred is a composition including (e.g., comprising, consisting essentially of, consisting of) one or more of beta acids, hexahydrobeta acids and tetrahydrobeta acids.

In another aspect, the invention provides a composition comprising a hop acid or hop acid derivative and a pharmaceutically acceptable carrier. The composition can further have an additional therapeutic agent. The compositions herein also include nutraceutical compositions comprising one or more of the aforementioned compound(s) and a nutraceutically acceptable carrier. The composition can include one or more additional nutraceutical agents.

Additional therapeutic agents include anti-inflammatory agents that are related to autoimmune diseases, pain, and diabetes. Such agents include salicylic acid, methyl salicylate, difulunisal, salsalate, olsalazine, sulfasalazine, acetanilide, acetaminophen, phenacetin, mefenamic acid, sodium meclofenamate, tolmetin, ketorolac, diclofenac, ibuprofen, naproxen, sodium daproxen, fenoprofen, ketoprofen, flurbioprofen, oxaprozin, piroxicam, meloxicam, tenoxicam, ampiroxicam, droxicam, pivoxicam, phenylbutazone, oxyphenbutazone, anitpyrine, aminopyrine, dipyrone, COX-2 selective non-steroidal anti-inflammatory drugs (NSAIDS) (e.g., celecoxib, rofecoxib, valdecoxib), nabumetone, apazone, nimensulide, indomethacin, sulindac, and etodolac, and anti-diabetic agents. A composition of the invention can further comprises a pharmaceutically acceptable carrier. Such a composition can be formulated for administration orally, topically, or rectally.

Another aspect is a method of treating a disease or disease symptom, including an inflammatory disease or disease symptom in a subject in need of such treatment (including identified as in need of such treatment) comprising administering to the subject an effective amount of a compound that includes any of the compounds herein.

In other aspects, the invention relates to a composition comprising a compound of any of the formulae herein, an additional therapeutic agent, and a pharmaceutically acceptable carrier. The additional therapeutic agent can be any one or more of an immunosuppressor (e.g., azathioprine, 6-mercaptopurine, cyclosporine A, tacrolimus, cyclophosphamide, and methotrexate), anti-inflammatory agent (e.g., sulfasalazine, olsalazine, mesalmine, ibuprofen, ketoprofen, piroxicam, naproxen sodium, sulindac, aspirin, choline subsalicylate, diflunisal, oxaproxin, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate), steroid (e.g., cortisone, budesonide, and prednisone), or antibiotics (e.g., tetracyclines, such as doxycycline, ciprofloxacin, azitliromycin, minocycline, clarithromycin and/or Augmentin, penicillins, such as penicillin G, penicillin V, methicillin, oxacillin, carbenicillin, nafcillin, ampicillin; cephalosporins, such as cefaclor, cefazolin, cefuroxime, moxalactam, carbapenems, monobactams, aminoglycosides, macrolides, lincomycins, polymyxins, sulfonamides, quinolones, cloramphenical, metronidazole, spectinomycin, trimethoprim, and vancomycin.

Yet another aspect of this invention relates to a method of treating a subject (e.g., mammal, human, horse, dog, cat) having or suffering from a disease or disease symptom related to an inflammatory pathology. The method includes administering to the subject (including a subject identified as in need of such treatment) an effective amount of a compound described herein, or a composition described herein to produce an anti-inflammatory effect. Subjects in need of such treatment are identified by a health care professional. This identification may be subjective (e.g., opinion) or objective (e.g., measurable by a test or diagnostic method).

Yet another aspect of this invention relates to a method of treating a subject (e.g., mammal, human, horse, dog, cat) having an inflammatory disease or disease symptom, including, but not limited to acne vulgaris, acute respiratory distress syndrome, Addison's disease, allergic rhinitis, allergic intraocular inflammatory diseases, ANCA-associated small-vessel vasculitis, ankylosing spondylitis, arthritis, asthma, atherosclerosis, atopic dermatitis, autoimmune hepatitis, autoimmune hemolytic anemia, autoimmune hepatitis, bacterial infection, Behcet's disease, Bell's palsy, bullous pemphigoid, cerebral ischaemia, chronic obstructive pulmonary disease, cirrhosis, Cogan's syndrome, contact dermatitis, chronic obstructive pulmonary disease, Crohn's disease, Cushing's syndrome, dermatomyositis, diabetes mellitus, discoid lupus erythematosus, eosinophilic fasciitis, erythema nodosum, exfoliative dermatitis, fibromyalgia, focal glomerulosclerosis, focal segmental glomerulosclerosis, giant cell arteritis, gout, gouty arthritis, graft-versus-host disease, hand eczema, Henoch-Schonlein purpura, herpes gestationis, hirsutism, idiopathic cerato-scleritis, idiopathic pulmonary fibrosis, idiopathic thrombocytopenic purpura, immune thrombocytopenic purpura, inflammatory bowel or gastrointestinal disorders, inflammatory dermatoses, lichen planus, lupus nephritis, lymphomatous tracheobronchitis, macular edema, multiple sclerosis, myasthenia gravis, myositis, nonspecific fibrosing lung disease, osteoarthritis, pancreatitis, pathogen infection, pemphigoid gestationis, pemphigus vulgaris, periodontitis, polyarteritis nodosa, polymyalgia rheumatica, pruritus scroti, pruritis/inflammation, psoriasis, psoriatic arthritis, pulmonary histoplasmosis, rheumatoid arthritis, relapsing polychondritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, shoulder tendinitis or bursitis, Sjogren's syndrome, Still's disease, stroke-induced brain cell death, Sweet's disease, systemic lupus erythematosus, systemic sclerosis, Takayasu's arteritis, temporal arteritis, toxic epidermal necrolysis, transplant-rejection and transplant-rejection-related syndromes, tuberculosis, type-1 diabetes, ulcerative colitis, uveitis, vasculitis, and Wegener's granulomatosis. The method includes administering to the subject (e.g., a subject identified as in need of such treatment) an effective amount of a compound described herein, or a composition described herein to produce such effect. Subjects in need of such treatment are identified by a health care professional. This identification may be subjective (e.g., opinion) or objective (e.g., measurable by a test or diagnostic method). The methods herein are also those wherein the subject is, in fact, treated, as shown by diagnostic test or opinion of subject or health care provider.

The methods also include a method of reducing an inflammatory response or chronic inflammation in a subject including administering to the subject (including a subject identified as in need of such treatment) an effective amount of a compound described herein, or a composition described herein to produce such effect. The method can include the steps of monitoring (assay, diagnostic test) inflammation either prior to, subsequent to, or both, administration of the compounds or compositions herein.

The methods also include a method of treating a metabolic syndrome in a subject including administering to the subject (including a subject identified as in need of such treatment) an effective amount of a compound described herein, or a composition described herein to produce such effect. In one embodiment, the composition is administered systemically. In another embodiment, the method reduces the risk of heart disease or diabetes. The method can include the steps of monitoring (assay, diagnostic test) a metabolic syndrome either prior to, subsequent to, or both, administration of the compounds or compositions herein.

In another aspect, the composition comprises a component having at least one compound that is a hop beta acid or hop beta acid derivative and a compound found in turmeric (e.g., curcumin). Particularly, a composition including (e.g., comprising, consisting essentially of, consisting of) one or more of a beta acid derivative (e.g., combinations of any two, three, four, or a combination) and a compound found in turmeric (e.g., curcumin).

The invention also relates to a method of making a compound described herein. The method includes any reactions or reagents or processes (including extraction, isolation, purification) as delineated in the schemes or examples herein. Alternatively, the method includes taking any one of the intermediate compounds described herein and reacting it with one or more chemical reagents in one or more steps to produce a compound described herein.

Also within the scope of this invention is a packaged product. The packaged product includes a container, one (or more) of the aforementioned compounds in the container, and a legend (e.g., a label or an insert) associated with the container and indicating administration of the compound for treating a disorder associated with inflammation.

In other embodiments, the compounds, compositions, and methods delineated herein are any of the compounds delineated herein or methods including them.

The details of one or more embodiments of the invention are set forth in the description below. Other features, objects, and advantages of the invention will be apparent from the description and from the claims.

### DETAILED DESCRIPTION

The present invention provides compositions comprising one or more hop acids or hop acid derivatives and processes for producing one or more hop acids or hop acid derivatives. These processes have improved efficiency for making isoalpha acids, rhoisoalpha acids, tetrahydroisoalpha acids, acids and hexahydroisoalpha acids, as well as for making beta acids, such as lupulone, colupulone, adlupulone and their derivatives. In particular embodiments, these beta acids include hexahydrobeta acids and tetrahydrobeta acids.

Exemplary rhoisoalpha acids that may be made by the process of the invention include, but are not limited to, any one or more of the following formulas:

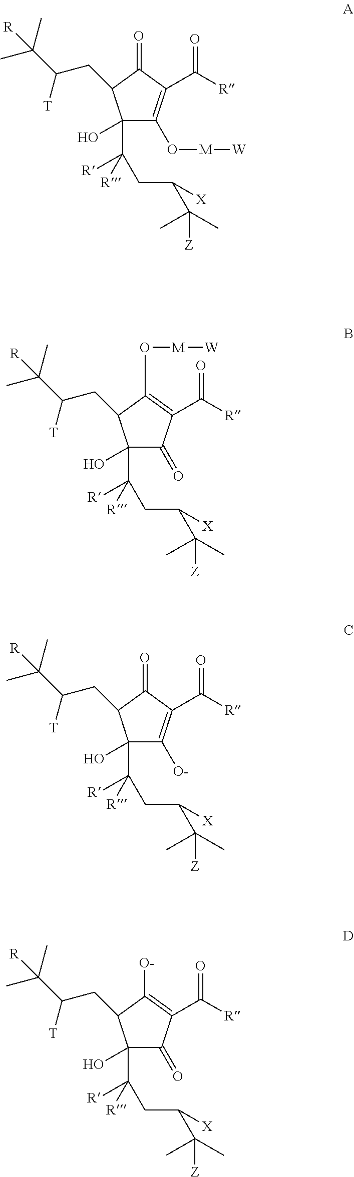

where R′ is selected from the group consisting of hydroxyl, OR and OCOR, R is independently alkyl, and R′″ is H; or R′ and R′″ taken together are ═O; and
where R″ is alkyl;
where R, T, X and Z are independently selected from the group consisting of H, F, Cl, Br, I and Pi-orbital, with the proviso that if one of R, T, X, or Z is a Pi orbital, then the adjacent R, T, X, or Z is also a Pi orbital, thereby forming a double bond; where M is magnesium or calcium;

where W is Cl, OH, SO4-, Br, I, Formula C or Formula D.

Exemplary beta acids that may be made by the process of the invention include, but are not limited to, any one or more of the following formulas:

any one or more of Formulas A, B, C and D:

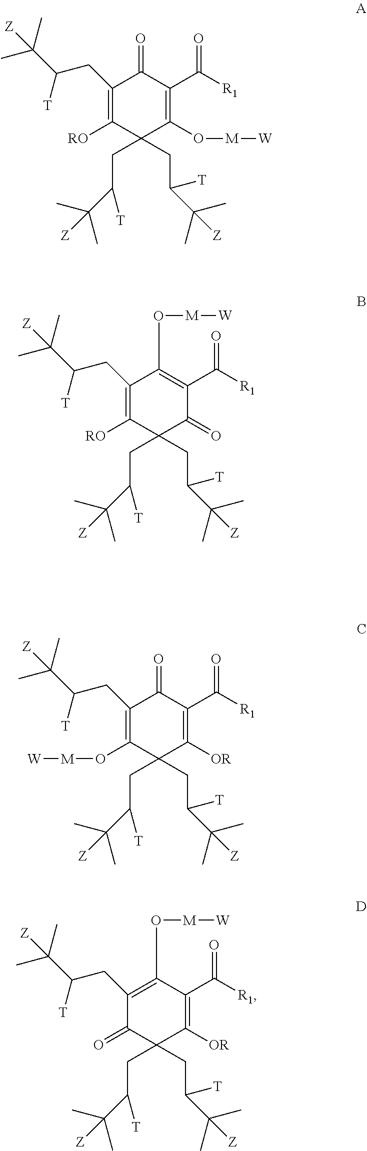

where R1 is alkyl;
where Z and T are independently selected from H and Pi-orbital with the proviso that if one of T or Z is a Pi orbital, then the adjacent T or Z is also a Pi orbital, thereby forming a double bond;
where M is magnesium or calcium;
where W is Cl, OH, SO4⁻, Br, I, or a compound of Formula E, F, G or H:

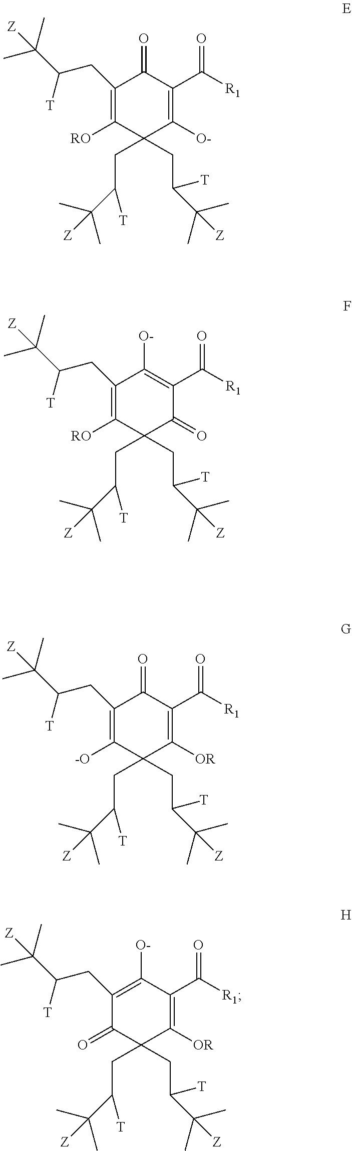

and where R is H, Na, K, Li or M-W. Advantageously, the magnesium salts of a beta acid are much less hygroscopic than the beta acids themselves.

In particular, the invention provides novel hop acid formulations having improved bioavailability, and the use of such compositions as anti-inflammatory agents, anti-metabolic syndrome compositions, and dietary supplements. Such compositions are useful for treating an inflammatory disease, a metabolic syndrome, a microbial infection or related disease symptoms.

In one aspect, the present invention provides methods for producing aqueous compositions containing between about 1% and 95%, inclusive, inorganic salts of beta acids, hexahydrobeta acids and tetrahydrobeta acids. Such aqueous formulations have improved bioavailability and are suitable for oral or topical administration to a subject. In addition, the production of such formulations is more efficient than prior art methods, and the aqueous formulations are more convenient to handle than prior art formulations.

As used herein, the term “isoalpha acid” refers to compounds isolated from hops plant products and which subsequently have been isomerized. The isomerization of alpha acids can occur thermally, for example, by boiling. Examples of isoalpha acids include, but are not limited to, isocohumulone, and isoadhumulone.

Examples of isoalpha acids include, but are not limited to, isohumulone, isocohumulone, and isoadhumulone.

As used herein, the term “rhoisoalpha acids” refers to alpha acids isolated from hops plant product and which subsequently have been isomerized and reduced (e.g., using sodium borohydride) including cis and trans forms. Examples of rhoisoalpha acids include, but are not limited to, rhoisohumulone, rhoisocohumulone, and rhoadhumulone.

As used herein, the term “tetrahydroisoalpha acids” refers to a class of reduced isoalpha acids produced by hydrogenation of isoalpha acids. Examples of tetrahydroisoalpha acids include, but are not limited to, tetrahydroisohumulone, tetrahydroisocohumulone and tetrahydroadhumulone.

As used herein, the term “hexahydroisoalpha acids” refers to a class of reduced isoalpha acids, produced by hydrogenation of isoalpha acids and sodium borohydride reduction of the resulting tetrahydroisoalpha acids. Examples of hexahydroisoalpha acids include, but are not limited to, hexahydroisohumulone, hexahydroisocohumulone and hexahydroadhumulone.

As used herein, the term “beta acids” refers to compounds that can be isolated from hops plant products, including but not limited to, lupulone, adlupulone, colupulone, tetrahydrolupulone, tetrahydroadlupulone, tetrahydrocolupulone and their derivatives. Exemplary beta acids include, but are not limited to, hexahydrobeta acids and tetrahydrobeta acids.

As used herein, the term “halo” refers to any radical of fluorine, chlorine, bromine or iodine.

The term “alkyl” refers to a hydrocarbon chain that may be a straight chain or branched chain, containing 1-20 or the indicated number of carbon atoms. For example, C₁-C₅ indicates that the group may have from 1 to 5 (inclusive) carbon atoms in it. The term “lower alkyl” refers to a C₁-C₆ alkyl chain.

The term “alkenyl” refers to a hydrocarbon chain that may be a straight chain or branched chain, containing 1-20 or the indicated number of carbon atoms and one or more double bonds in the chain (e.g., propylenyl, isopentylenyl). For example, C₁-C₁₀ indicates that the group may have from 1 to 10 (inclusive) carbon atoms in it.

The term “arylalkyl” refers to a moiety in which an alkyl hydrogen atom is replaced by an aryl group. The term “cycloalkylalkyl” refers to a moiety in which an alkyl hydrogen atom is replaced by a cycloalkyl group.

The term “aryl” refers to an aromatic monocyclic, bicyclic, or tricyclic ring system having carbon ring atoms, wherein 0, 1, 2, 3, or 4 atoms of each ring may be substituted by a substituent.

The term “cycloalkyl” as employed herein includes saturated and partially unsaturated cyclic hydrocarbon groups having 3 to 12 carbons, preferably 3 to 8 carbons, and more preferably 3 to 6 carbons.

The term “leaving group” is any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

As used herein the term “substituent” or “substituted” means that a hydrogen radical on a compound or group (such as, for example, alkyl, alkenyl, alkynyl, alkylene, aryl, aralkyl, heteroaryl, heteroaralkyl, cycloalkyl, cyclyl, heterocycloalkyl, or heterocyclyl group) is replaced with any desired group that do not substantially adversely affect the stability of the compound. In one embodiment, desired substituents are those which do not adversely affect the activity of a compound. The term “substituted” refers to one or more substituents (which may be the same or different), each replacing a hydrogen atom.

Examples of substituents include, but are not limited to, halogen (F, Cl, Br, or I), hydroxyl, amino, alkylamino, arylamino, dialkylamino, diarylamino, cyano, nitro, mercapto, oxo (i.e., carbonyl), thio, imino, formyl, carbamido, carbamyl, carboxyl, thioureido, thiocyanato, sulfoamido, sulfonylalkyl, sulfonylaryl, alkyl, alkenyl, alkoxy, mercaptoalkoxy, aryl, heteroaryl, cyclyl, heterocyclyl, wherein alkyl, alkenyl, alkyloxy, aryl, heteroaryl, cyclyl, and heterocyclyl are optionally substituted with alkyl, aryl, heteroaryl, halogen, hydroxyl, amino, mercapto, cyano, nitro, oxo (═O), thioxo (═S), or imino (═NR), where R is as defined herein.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Reduced isoalpha acids and beta acids can be prepared by purification from natural hops and also chemical synthesis according to traditional methods. The compounds delineated herein can be synthesized using conventional methods known in the art.

The term “extract” refers to a concentrated preparation of the essential constituents of a plant (e.g., medicinal plant, hops). Typically, an extract is prepared by drying and powderizing the plant. Optionally, the plant, the dried plant or the powderized plant may be boiled in solution. The extract may be used in liquid form, or it may be mixed with other liquid or solid herbal extracts. Alternatively, the herbal extract may be obtained by further precipitating solid extracts from the liquid form. Edible plant extracts include those from any plant that is edible to a human (e.g., fruit extract, vegetable extract, root extract, leaf extract, tree or bark extract, bean extract, and the like) and includes, for example, green tea extract, red onion extract, grape seed extract, cocoa extract, red clover extracts, and soy extracts.

An extract can be prepared by drying and subsequently cutting or grinding the dried material. The extraction process may then be performed with the help of an appropriate choice of solvent, typically supercritical or liquid carbon dioxide, ethanol/water mixture, methanol, butanol, iso-butanol, acetone, hexane, petroleum ether or other organic solvents by means of maceration, percolation, repercolation, counter-current extraction, turbo-extraction, or by carbon-dioxide supercritical (temperature/pressure) extraction. The extract may then be further evaporated and thus concentrated to yield by means of air drying, spray drying, vacuum oven drying, fluid-bed drying or freeze-drying, the extract product.

The synthesized compounds can be separated from a reaction mixture and further purified by a method such as column chromatography, high pressure liquid chromatography, or recrystallization. As can be appreciated by the skilled artisan, further methods of synthesizing the compounds herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T.W. Greene and P.G.M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); L. Paquette, ed., Encyclopedia of Reagents for Organic Synthesis, John Wiley and Sons (1995), and M. Verzele and D. De Keukeleire, Chemistry and Analysis of Hop and Beer Bitter Acids, Elsevier (1991), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg. Effective doses will also vary depending on route of administration, as well as the possibility of co-usage with other agents.

An “anti-inflammatory” compound reduces inflammation. Reducing inflammation refers to decreasing, ameliorating or inhibiting an inflammatory response. One skilled in the art can readily recognize a reduction in a sign or symptom associated with an inflammatory response.

The morbidity associated with asthma and allergic rhinitis is associated with inflammation that occurs in the lungs and nasal passages. Allergies are associated with a hypersensitive immune response generated against allergens that do not normally generate an immune response. Methods of the invention are useful for the treatment of allergies, allergic rhinits, and associated inflammation.

Inflammation may be assessed using any method known in the art. In one example, inflammation is assessed by detecting pro-inflammatory markers or the release of pro-inflammatory molecules (e.g., IL-12, TNF-alpha, IL-1 beta, IL-6, IL-10, GRO-CINC-1, IL-5, IL-18 or MCP-1), or the activation of pro-inflammatory signaling. The detection of such molecules may be determined in vitro (by Western or Northern analysis, for example) or in vivo (as measured by immunohistochemical methods). Alternatively, inflammation may be determined using assays that measure myeloperoxidase activity, which is an indication of acute inflammation. Optionally, inflammation is detected by assessing the overall morphology of tissues or the detection of infiltration of pro-inflammatory cells, such as leukocytes, monocytes, macrophages (F4/80, or ER-MP20 for example), lymphocytes (IgA, IgG, IgM, CD4 and CD8 staining), neutrophils, and eosinophils (by immunohistochemical methods).

Metabolic syndrome is a cluster of heart disease and diabetes risk factors that occur together and increase a patient's risk for serious disease, including heart disease, stroke and diabetes. The criteria for metabolic syndrome include an increased waist circumference (abdominal obesity), elevated triglycerides, reduced high-density lipoprotein cholesterol (HDL-C), elevated blood pressure, and/or an elevated fasting glucose. In particular, levels of triglycerides of 150 mg/dL or higher; a high density lipoproteins (HDL) cholesterol lower than 40 mg/dL for men and lower than 50 mg/dL for women; a blood pressure level of 130/85 mm Hg or higher; or a fasting glucose of 100 mg/dL or higher. For most Americans, a waist circumference of 35 inches or more for women and 40 inches or more for men is considered abnormally increased. An individual who has abnormal levels of at least three of the listed criteria is considered to have metabolic syndrome. Many physicians believe that metabolic syndrome is likely associated with resistance to insulin. Metabolic syndrome increases the risk for atherosclerotic cardiovascular disease by 1.5-3 fold, and raises the risk for type 2 diabetes by 3-5 fold. It affects over 26 percent of adults, or over 50 million Americans.

Clinical management of metabolic syndrome is focused on reducing the risk for atherosclerotic cardiovascular disease, and the risk of type 2 diabetes in patients who have not yet developed clinical diabetes. Recently published results indicate that one in five adults in the U.S. has metabolic syndrome. Current methods of treating metabolic syndrome are inadequate. Compositions of the invention comprising beta acids made by the methods described herein are useful for the prevention or treatment of a metabolic syndrome, or for the prevention or treatment of any one or more of the risk factors associated with a metabolic syndrome.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385;

The compounds of this invention may be modified by appending appropriate functionalities to enhance selective biological properties. Such modifications are known in the art and include those which increase biological penetration into a given biological compartment (e.g., blood, lymphatic system, nervous system), increase oral availability, increase solubility to allow administration by injection, alter metabolism and alter rate of excretion.

Acceptable salts of the compounds, including pharmaceutically acceptable salts, of this invention include those derived from acceptable inorganic and organic acids and bases. Examples of suitable acid salts include acetate, adipate, alginate, aspartate, benzoate, benzenesulfonate, bisulfate, bromide, butyrate, citrate, camphorate, camphorsulfonate, chloride, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, hydroxyl, iodide, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic may be employed in the preparation of salts useful as intermediates in obtaining the compounds of the invention and their pharmaceutically acceptable acid addition salts. Salts derived from appropriate bases include any monovalent cation, including but not limited to lithium, sodium, potassium, silver, copper, or divalent cation, including but not limited to magnesium, calcium, barium, chromium, manganese, iron, cobalt, nickel, copper, zinc, and cadmium. In particular embodiments, the salt is derived from alkali metals (e.g., sodium, potassium, lithium), alkaline earth metal (e.g., magnesium, calcium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The compounds of the formulae described herein can, for example, be administered orally, buccally, nasally, rectally, transmucosally, topically, in an ophthalmic preparation, by injection subdermally, intraperitoneally, intramuscularly, or subcutaneously; or by inhalation, with a dosage ranging from about 0.5 to about 100 mg/kg of body weight, alternatively dosages between 1 mg and 1000 mg/dose, every 4 to 120 hours, or according to the requirements of the particular drug; or any dosage range in which the low end of the range is any amount between 0.1 mg/day and 400 mg/day and the upper end of the range is any amount between 1 mg/day and 500 mg/day (e.g., 5 mg/day and 95 mg/day, 100 mg/day and 500 mg/day); or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 5 mg/kg/day, 25 mg/kg/day and 75 mg/kg/day). The methods herein contemplate administration of an effective amount of compound or compound composition to achieve the desired or stated effect. Typically, the pharmaceutical compositions of this invention will be administered from about 1 to about 6 times per day. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound. In one aspect, the dosage in clinical or nutraceutical use is normally within a range of 0.05 g-3 g per adult per day for beta acid derivatives, such as beta acids, hexahydrobeta acids and tetrahydrobeta acids, or any beta acid derivative. Alternatively, alpha acids or alpha acid derivatives are used.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including inflammatory disorders or symptoms thereof. References which include examples of additional therapeutic agents are: 1) Burger's Medicinal Chemistry & Drug Discovery 6^{th} edition, by Alfred Burger, Donald J. Abraham, ed., Volumes 1 to 6, Wiley Interscience Publication, NY, 2003.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically and nutraceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms, such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins, such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, by inhalation spray, topically, rectally, nasally, buccally, vaginally or via an implanted reservoir, preferably by oral administration or administration by injection. The pharmaceutical compositions of this invention may contain conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intramuscular, intra-articular, intrasynovial, intrasternal, intrathecal, intralesional and intracranial injection.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents that are commonly used in the formulation of pharmaceutically acceptable dosage forms, such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs) (Negrin et al., Biomaterials, 22(6):563, 2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. Another embodiment is a compound of any of the formulae herein made by a process delineated herein, including the processes exemplified in the schemes and examples herein. Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a disease, disorder or symptom thereof delineated herein.

The preparations containing beta acids are manufactured by an ordinary method using ordinary recipients and food additives. As an oral preparation, it can be formulated in the form of ordinary tablets, capsules, fine granules or powders. It also can be added to a food product as a pure form or extracted hops. The food product can be a solid, a paste, or a liquid food product, such as milk, tea, soft drinks, juices, coffee, seasonings, cereals, water, cookies, yogurt, chewing gum, chocolate, or soups. The food product can be a “non-alcoholic” food product, that is a food product having low (e.g., <3%, <2%, <1%, <0.5%, <0.25% <0.1%, <0.05%) or no (e.g., essentially zero) alcohol content. In the invention, the nutraceutical carrier for the compositions herein may include, a base of fruit, vegetables or fruit or vegetable juice or puree, a base of vegetable soup or bouillon, a soy-milk drink, a tea or coffee drink, or a nutritive supplement.

Additionally the components can be fortified with electrolytes, flavors, other plant extracts, preservatives, and other additives, (e.g., vitamin supplements and maltodextrin). Examples of preservatives include, but are not limited to, ascorbic acid and propyl gallate. Examples of electrolytes include, but are not limited to, magnesium sulfate and potassium chloride.

The invention will be further described in the following examples. It should be understood that these examples are for illustrative purposes only and are not to be construed as limiting this invention in any manner.

### Examples

In general, inorganic salts of hop acith are prepared as follows. An aqueous solution or resinous preparation of one or more hop acid including the hop acids known as beta acids, alpha acids, isoalpha acids, rhoisoalpha acids, tetrahydroisoalpha acids, hexahydroisoalpha acids, or any mixture thereof, is prepared by adding water and a suitable base (e.g., NaOH, KOH) to the hop acid mixture until the pH of the hop acid mixture is between 7 and 12 and the concentration of the hop acid mixture is between 50% and 1%. The aqueous solution or resinous preparation is then mixed with either a water slurry of the inorganic metal salts or an aqueous preparation of those salts, including but not limited to potassium salts, iron salts, calcium salts, lithium salts, or zinc salts, depending upon the solubility of the metal salts to be used.

Several examples are included to illustrate this process, but these examples are not meant to be all inclusive of methods or preparations, which may vary by the type of metal salt, the hop acid or mixture of hop acids used, and varied concentrations. Examples are included to illustrate the varied possible ways of producing these metal salts. Differing formulations of various hop acids, quantities of water, types of metal salts used, and drying techniques are considered interchangeable.

### Example 1

### Preparation of a Rhoisoalpha Acid Salts

An inorganic salt of rhoisoalpha acids is produced using any standard methods known in the art. In one embodiment, a rhoisoalpha acid is produced according to the following method.

An empty drum was placed on a scale and tared. To the drum was added 80 kg of a mixture of rhoisoalpha acids (30%) in deionized water (75 L) at room temperature. The mixture was subjected to gentle agitation to form an aqueous slurry. MgSO₄ (45 kg) was added to the slurry at one time and the agitation was continued for 5-10 minutes until the MgSO₄ was homogeneously distributed. After 10 minutes, a small sample was removed to determine whether the reaction had reached completion. This was determined using an HPLC to assay the presence of rhoisoalpha acids magnesium salt. When the reaction was complete, the mixture was removed and deionized water was added to adjust the concentration of rhoisoalpha acids magnesium salt to 15-17% having 83-85% water content. The mixture was then dried using standard methods. When the drying was completed, the flaky products were packed in aluminum coated polyethelene bags, heat sealed and stored at room temperature prior to analysis.

### Example 2

### Preparation of Beta Acids Magnesium Salts

Beta acids magnesium salts were prepared as follows. 5000 ml of hop beta acids solution containing approximately 500 g of beta acids potassium salt was stirred at room temperature. The pH of the solution was adjusted to pH 11.50 by the drop wise addition of 100 ml of 20% KOH solution. The solution was then diluted with deionized water (1100 ml) while the pH was maintained at 11.50.

840 ml of 10% MgSO₄ solution was added to the solution under vigorous stirring at room temperature. After the addition, the mixture was stirred for 30 minutes then the white precipitate was filtered through a Buchner funnel using Whatman #45 filter paper. The precipitate was washed with deionized water and dried to get 490 g of beta acids magnesium salt (purity: >95% as the magnesium content).

5000 ml of hop beta acid solution that contained approximately 500 g of beta acid potassium salt was stirred at room temperature. The solution's pH was adjusted to pH 11.50 by the drop wise addition of 100 ml of 20% KOH solution. The solution was then diluted with deionized water (1100 ml) while the pH was maintained at 11.50.

840 ml of 10% of MgSO4 solution was added to the solution under vigorous stirring at room temperature. After the addition, the mixture was stirred for 30 minutes then the mixture was evaporated to obtain 560 g of a pale yellow solid. (purity:>70%).

### Example 3

### Preparation of Tetrahydrobetaacids Magnesium Salts

Tetrahydrobetaacids magnesium salts were obtained as follows. 1.25 liters of an aqueous alkaline solution containing 20% tetrahydrobeta acids was blended with 1.25 liter of an aqueous 6M potassium carbonate solution at room temperature. After stirring for 30 minutes, the solution's pH was adjusted to pH 11.50 by the drop wise addition of 50 ml of 20% KOH solution. The solution was then diluted with deionized water (550 ml) while maintaining the solution pH at 11.50. 420 ml of 10% of MgSO4 solution was added to the solution under vigorous stirring at room temperature. After the addition, the mixture was stirred for 30 minutes then the white precipitate was filtered through a Buchner funnel using Whatman #45 filter paper and washed with deionized water and dried to get 220 g of beta acid magnesium salt (purity:>95% as the magnesium content).

### Example 4

### Preparation of Rhoisoalpha Acids Iron Salts

To prepare the iron salt of rhoisoalpha acid, 300 grams of an aqueous 30% rhoisoalpha acid solution having a pH of 10 was mixed with 70 grains of FeSO4-7H₂O, which had been mixed previously with 500 mL deionized water. This slurry was mixed until homogeneous. The slurry was then poured directly onto a drying tray and dried.

### Example 5

### Preparation of Rhoisoalpha Acids Calcium Salts

To prepare the calcium salt of rhoisoalpha acid, 300 grams of an aqueous 30% rhoisoalpha acid solution having a pH of 11 was mixed with 37 grams of CaCl₂-2H₂O, which had been mixed previously with 200mL deionized water. This slurry was mixed until homogeneous. The slurry was then poured directly onto a drying tray and dried.

### Example 6

### Preparation of Rhoisoalpha Acids Lithium Salts

To prepare the lithium salt of rhoisoalpha acid, 300 grams of an aqueous 30% rhoisoalpha acid solution having a pH of 9 was mixed with 21 grams of LiOH—H₂O, which had been mixed previously with 300 mL deionized water. This slurry was mixed until homogeneous. The slurry was then filtered through a Buchner funnel to remove excess water and placed onto a drying tray and dried.

### Example 7

### Preparation of Rhoisoalpha Acids Calcium Salts

To prepare the calcium salt of rhoisoalpha acid, 300 grams of an aqueous 30% rhoisoalpha acid solution having a pH of 11 was mixed with 37 grams of CaCl₂-2H₂O, which had been mixed previously with 200 mL deionized water. This slurry was mixed until homogeneous. The slurry was then poured directly onto a drying tray and dried.

### Example 8

### Preparation of Rhoisoalpha Acids Zinc Salts

To prepare the zinc salt of rhoisoalpha acid, 300 grams of an aqueous 30% rhoisoalpha acid solution having a pH of 8 was mixed with 72 grams of ZnSO₄-7H₂O, which had been mixed previously with 500 mL deionized water. This slurry was mixed until homogeneous. The slurry was then poured directly onto a drying tray and dried.

### Example 9

### Preparation of Rhoisoalpha Acids Potassium Salts

To prepare the potassium salt of rhoisoalpha acid, 300 grams of an aqueous 30% rhoisoalpha acid solution having a pH of 10 was mixed with 35 grams of K₂CO₃ which had been mixed previously with 300 mL deionized water. This slurry was mixed until homogeneous. The slurry was then poured directly onto a drying tray and dried.

### Example 10

### Preparation of Tetrahydroisoalpha Acid Calcium Salts

To prepare the calcium salt of tetrahydroisoalpha acid, 1000 grams of an aqueous 9% tetrahydroisoalpha acid solution having a pH of 10.5 was mixed with 42 grams of CaCl₂-2H₂O, which had been mixed previously with 100 mL deionized water. This slurry was mixed until homogeneous. The slurry was then filtered through a Buchner funnel to remove excess water and placed onto a drying tray and dried.

### Example 11

### Preparation of Tetrahydroisoalpha Acid Zinc Salts

To prepare the zinc salt of a mixture of tetrahydroisoalpha acid and hexahydroisoalpha acid, 450 grams of an aqueous 10% hexahydroisoalpha and 450 grams of an aqueous 10% tetrahydroisoalpha solution having a pH of 10 was mixed with 73 grams of ZnSO₄-7H₂O, which had been mixed previously with 200 mL deionized water. This slurry was mixed until homogeneous. The slurry was then filtered through a Buchner funnel to remove excess water and placed onto a drying tray and dried.

Virtually any inorganic salt (e.g., magnesium, calcium, potassium, iron, lithium, or zinc salt) may be used in the process set forth above. In one embodiment, the salt is a magnesium salt (e.g., magnesium sulfate), is an iron salt (e.g., FeSO4), is a calcium salt (e.g., CaCl₂), is a lithium salt (e.g., LiOH), is a zinc salt (e.g., ZnSO₄), is a potassium salt (e.g., ZnSO₄), or is a potassium salt (e.g., K₂CO₃). In yet other embodiments of any of the above aspects, the inorganic salt/hop acid molar ratio, magnesium/rhoisoalpha acids or calcium/rhoisoalpha acids molar ratio is in a range between 0.3 and 0.8 (e.g., 0.3, 0.4, 0.5, 0.6, 0.7, 0.8), inclusive. In one embodiment of any of the above aspects, the magnesium/rhoisoalpha acids or calcium/ rhoisoalpha acids molar ratio is in a range between 0.4 and 0.6. In related embodiments, the lower limit of the range is any number between 0.3 and 0.79 and the upper limit of the range is any number between 0.35 and 0.8.

As set forth in the above examples, the invention provides processes for producing a solid salt of alpha acids or beta acids. Virtually any isoalpha acids, rhoisoalpha acids, tetrahydroisoalpha acids, hexahydroisoalpha acids, beta acids, hexahydrobeta acids, tetrahydrobeta acids, lupulone, colupulone, adlupulone or derivatives thereof may be used in the processes of the invention. In one embodiment, the concentration of rhoisoalpha acids or beta acids present in the aqueous solution ranges between 5% and 50%, inclusive. In other embodiments, the concentration ranges between 5-45% (e.g., 9%, 10%, 15%, 20%, 25%, 30%, 35%, 40%, and 45%), inclusive. In yet other embodiments, the lower end of the range is any number between 9 and 49%; and the upper end of the range is any number between 10 and 50%. The slurry may be dried to obtain an inorganic salt (e.g., magnesium or calcium) of rhoisoalpha acids using any standard method or combination of methods, including but not limited to, spray drying, vaccum drying, drum drying, pan drying, window drying and freeze drying.

This process provides advantages over previous methods for producing solid salts of hop acids, such as those described in U.S. Pat. No. 5,624,701, which require a four step process that includes heating an aqueous alkaline solution of a hop acid with an aqueous salt solution to produce a solid salt of a hop acid. This heating step accelerates the degradation of alpha acids during salt formation and is undesirable.

Advantageously, the present method does not require a heating step, but is carried out at room temperature. The term “room temperature” means between 15° C. and 25° C. (e.g., 15, 16, 17, 18, 19, 20, 21, 22, 23, 24, or 25° C.), where the lower end of the range is any number between 15 and 24; and the upper end of the range is any number between 16 and 25.

In addition, prior methods required using hop acids at concentrations between 4% and 7% inclusive during the magnesium salt formation reaction. These low concentrations of hop acids increase the time required for the reaction, and increase costs. The present invention uses reduced isoalpha acid at concentrations between 9% and 50% (e.g., 9, 10, 15, 20, 25, 30, 35, 45, and 50%), inclusive. These higher concentrations allow the reaction to proceed 5-10 times more quickly than previously described methods, which advantageously reduces labor, energy, and other production costs.

Compounds are prepared in a manner essentially as described above and in the general schemes. The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. Another embodiment is a compound of any of the formulae herein made by a process delineated herein, including the processes exemplified in the schemes and examples herein. Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a disease, disorder or symptom thereof delineated herein.

All references cited herein, whether in print, electronic, computer readable storage media or other form, are expressly incorporated by reference in their entirety, including but not limited to, abstracts, articles, journals, publications, texts, treatises, interne web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising one or more compounds of Formula A, B, C and D: 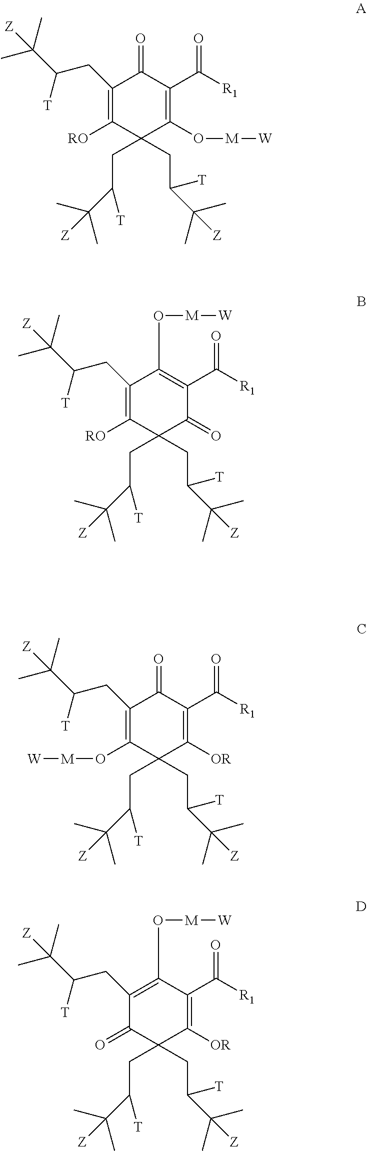
where R1 is alkyl;
where Z and T are independently selected from H and Pi-orbital, with the proviso that if one of T or Z is a Pi orbital, then the adjacent T or Z is also a Pi orbital, thereby forming a double bond;
where M is a monovalent or divalent cation selected from the group consisting of lithium, sodium, potassium, silver, copper, magnesium, calcium, barium, chromium, manganese, iron, silver, cobalt, nickel, copper, zinc, and cadmium;
where W is absent or is Cl, OH, SO4-, Br, I, Formula E, F, G or H: 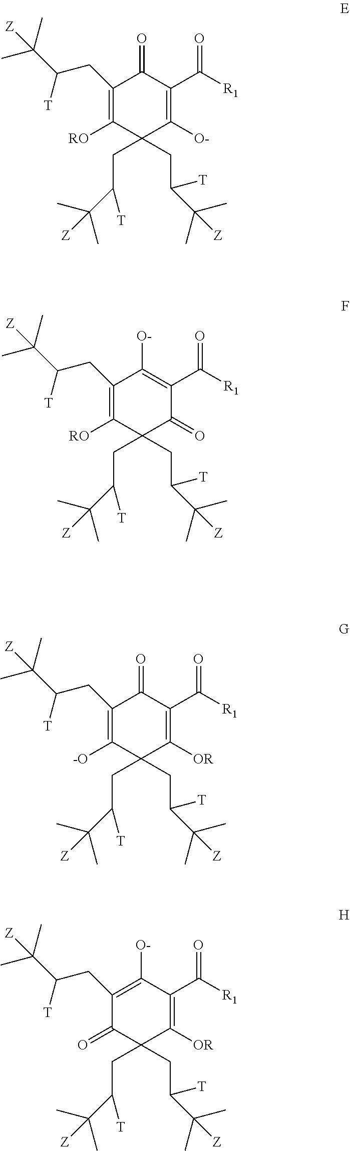
and where R is H, Na, K, Li or M-W.

2. A composition comprising an aqueous composition comprising 1-95% a combination of at least two of Formula A, B, C and D: 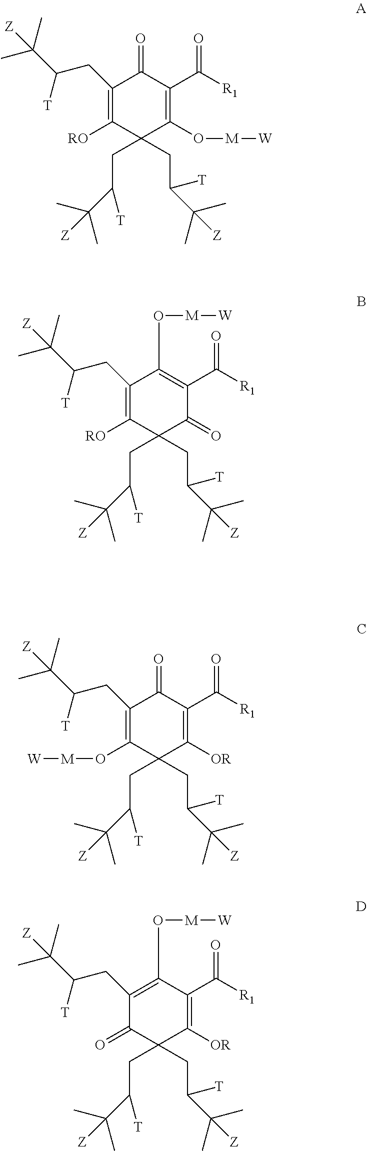
where R1 is alkyl;
where Z and T are independently selected from H and Pi-orbital, with the proviso that if one of T or Z is a Pi orbital, then the adjacent T or Z is also a Pi orbital, thereby forming a double bond;
where M is lithium, sodium, potassium, silver, copper, magnesium, calcium, barium, chromium, manganese, iron, silver, cobalt, nickel, copper, zinc, or cadmium;
where W is absent or is Cl, OH, SO4-, Br, I, Formula E, F, G or H: 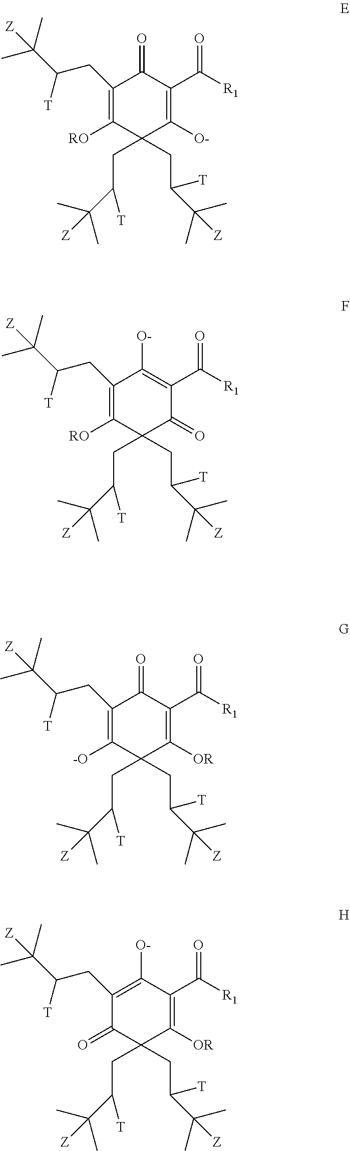
and where R is H, Na, K, Li or M-W.

3. The composition of claim 2, wherein the aqueous composition comprises beta acids, hexahydrobeta acids or tetrahydrobeta acids.

4. The composition of claim 2, wherein RI is isopropyl, isobutyl or sec-butyl.

5. The composition of claim 2, wherein the pH of the aqueous composition is between 7.0 and 10.0.

6. The composition of claim 5, wherein the pH of the aqueous composition is between 7.0 and 9.5.

7. The composition of claim 6, wherein the pH of the aqueous composition is between 7.0 and 8.0.

8. The composition of claim 7, wherein the pH of the aqueous composition is between 7.2 and 7.4.

9. The composition of claim 2, wherein the water content of the aqueous composition is between 1-20%.

10. The composition of claim 2, wherein the water content of the aqueous composition is between 1-10%.

11. The composition of claim 2, wherein the content of the salt composition is between 20-90%.

12. A composition comprising one or more compounds of 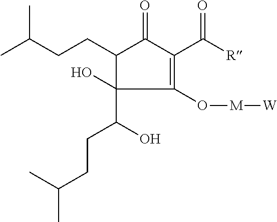
where R″ is alkyl;
where M is lithium, sodium, potassium, silver, copper, magnesium, calcium, barium, chromium, manganese, iron, silver, cobalt, nickel, copper, zinc, or cadmium; and
where W is absent or is Cl, OH, SO4-, Br, or I.

13. A process for the production of an inorganic salt of a hop acid, the method comprising:
(a) providing an aqueous solution containing 10-50% of hop acid, wherein the solution is at room temperature;
(b) adding an inorganic salt to the aqueous solution with agitation to form a slurry, wherein the slurry is at room temperature;
(c) mixing until the slurry is homogeneous; and
(d) drying the slurry to obtain an inorganic salt of an hop acid.

14. A process for the production of an inorganic salt of an isoalpha acid, the method comprising:
(a) providing an aqueous alkaline solution containing 10-50% of an isoalpha acid, wherein the solution is at room temperature;
(b) adding an inorganic salt to the aqueous alkaline solution with agitation to form a slurry, wherein the slurry is at room temperature;
(c) mixing until the shiny is homogeneous; and
(d) drying the slurry to obtain an inorganic salt of an isoalpha acid.

15. A process for the production of an inorganic salt of a beta acid, the method comprising:
(a) providing an aqueous alkaline solution containing 10-50% of beta acid, wherein the solution is at room temperature;
(b) adding an inorganic salt to the aqueous alkaline solution with agitation to form a slurry, wherein the slurry is at room temperature;
(c) mixing until the slurry is homogeneous; and
(d) drying the slurry to obtain an inorganic salt of an isoalpha acid.

16. The process of any one of claims 14-16, wherein the inorganic salt is a lithium, sodium, potassium, silver, copper, magnesium, calcium, barium, chromium, manganese, iron, silver, cobalt, nickel, copper, zinc, or cadmium salt.

17. The process of any one of claims 14-16, wherein the inorganic salt is a magnesium, calcium, potassium, lithium, iron, or zinc salt.

18. The process of claim 14 or 15, wherein the hop acid is an isoalpha acid selected from the group consisting of isoalpha acids, rhoisoalpha acids, tetrahydroisoalpha acids, and hexahydroisoalpha acids and derivatives or mixtures thereof.

19. The process of claim 14 or 15, wherein the hop acid is a beta acid selected from the group consisting of lupulone, colupulone, adlupulone and derivatives or mixtures thereof.

20. The process of claim 14 or 15, wherein the beta acid is a hexahydrobeta acids or tetrahydrobeta acids.

21. The process of any one of claims 14-16, wherein the method further comprises the step of filtering the homogenous slurry of step (c) prior to step (d).

22. A process for the production of a magnesium salt of an isoalpha acid or a rhoisoalpha acid, the method comprising:
(a) providing an aqueous solution containing 10-50% of an isoalpha acid or a rhoisoalpha acid, wherein the solution is at room temperature;
(b) adding an inorganic magnesium salt to the aqueous alkaline solution with agitation to form a slurry, wherein the slurry is at room temperature;
(c) mixing until the slurry is homogeneous; and
(d) drying the slurry to obtain a magnesium salt of an isoalpha acid or a rhoisoalpha acid.

23. The process of claim 22, wherein the aqueous solution is an aqueous alkaline solution.

24. The process of claim 22, wherein the magnesium salt is magnesium sulfate.

25. A process for the production of a calcium salt of an isoalpha acid or a reduced isoalpha acid, the method comprising:
(a) providing an aqueous solution containing 10-50% of an isoalpha acid or a reduced isoalpha acid, wherein the solution is at room temperature;
(b) adding an inorganic calcium salt to the aqueous solution with agitation to form a slurry, wherein the slurry is at room temperature;
(c) mixing until the slurry is homogeneous; and
(d) drying the slurry to obtain a calcium salt of an isoalpha acid or a reduced isoalpha acid.

26. The process of claim 25, wherein the aqueous solution is an aqueous alkaline solution.

27. The process of claim 25, wherein the calcium salt is at least one of calcium carbonate, calcium chloride, or calcium hydroxide.

28. The process of any one of claim 14, 15, or 17-27, wherein the concentration of isoalpha acids or of rhoisoalpha acids present in the aqueous solution is between 10% and 45%.

29. The process of claim 28, wherein the concentration of an isoalpha acids or of rhoisoalpha acids present in the aqueous solution is between 15% and 45%. 10

30. The process of claim 29, wherein the concentration of an isoalpha acids or of rhoisoalpha acids present in the aqueous solution is 15%.

31. The process of any one of claim 14, 15, or 17-27, wherein the magnesium/isoalpha acids or rhoisoalpha acids or calcium/isoalpha acids or rhoisoalpha acids molar ratio is in a range between 0.3 and 0.8.

32. The process of any one of claim 14, 15, or 17-27, wherein the drying is accomplished by a method selected from the group consisting of spray drying, vacuum drying, drum drying, pan drying, window drying and freeze drying, or any combination thereof.

33. The process of any one of claim 14, 15, or 17-27, wherein the reduced isoalpha acid is selected from the group consisting of tetrahydroisoalpha acids and hexahydro-isoalpha acids.

34. The process of any one of claim 14, 15, or 17-27, wherein room temperature is between 15° C. and 25° C.

35. The process of any one of claim 14, 15, or 17-27, wherein the method further comprises the step of filtering the homogenous slurry of step (c) prior to step (d).

36. A reduced isoalpha acid or inorganic salt of a reduced isoalpha acids made by the process of any one of claims 14-35.

37. An inorganic salt of a reduced isoalpha acid, wherein the isoalpha acid is selected from the group consisting of rhoisoalpha acids, tetrahydroisoalpha acids, and hexahydroisoalpha acids.

38. The inorganic salt of claim 37, wherein salt comprises a monovalent or divalent cation.

39. The inorganic salt of claim 38, wherein the monovalent cation is selected from the group consisting of lithium, sodium, potassium, silver, copper.

40. The inorganic salt of claim 38, wherein the divalent cation is magnesium, calcium, barium, chromium, manganese, iron, cobalt, nickel, copper, zinc, cadmium.

41. A process for the production of a magnesium salt of beta acids, the method comprising:
(a) providing an aqueous alkaline solution containing 10-50% of a beta acids, wherein the solution is at room temperature;
(b) adding an inorganic magnesium salt to the aqueous alkaline solution with agitation to form a slurry, wherein the slurry is at room temperature;
(c) mixing until the slurry is homogeneous; and
(d) drying the sluny to obtain a magnesium salt of a beta acids.

42. The process of claim 41, wherein the magnesium salt is magnesium sulfate.

43. A process for the production of a calcium salt of beta acids, the method comprising:
(a) providing an aqueous alkaline solution containing 10-50% of a beta acids, wherein the solution is at room temperature;
(b) adding an inorganic calcium salt to the aqueous alkaline solution with agitation to form a slurry, wherein the slurry is at room temperature;
(c) mixing until the slurry is homogeneous; and
(d) drying the slurry to obtain a calcium salt of a beta acids.

44. The process of claim 43, wherein the calcium salt is at least one of calcium carbonate, calcium chloride, or calcium hydroxide.

45. The process of any one of claims 41-45, wherein the concentration of beta acids present in the aqueous alkaline solution is between 10% and 45%.

46. The process of claim 45, wherein the concentration of a beta acids in the aqueous alkaline solution is between 15% and 45%.

47. The process of claim 46, wherein the concentration of beta acids present in the aqueous alkaline solution is 20%.

48. The process of any one of claims 41-47, wherein the magnesium/beta acids or calcium/beta acids molar ratio is in a range between 0.3 and 0.8.

49. The process of any one of claims 41-47, wherein the method further comprises the step of filtering the homogenous slurry of step (c) prior to step (d).

50. The process of any one of claims 41-47, wherein the drying is accomplished by a method selected from the group consisting of spray drying, vacuum drying, drum drying, pan drying, window drying and freeze drying, or any combination thereof.

51. The process of any one of claims 41-47, wherein the beta acids is selected from the group consisting of tetrahydrobeta acids, and hexahydrobeta acids.

52. The process of any one of claims 41-47, wherein room temperature is between 15° C. and 25° C.

53. Beta acids made by the process of any one of claims 35-46.

54. An inorganic salt of a beta acid, wherein the beta acid is selected from the group consisting of lupulone, colupulone, adlupulone, hexahydrobeta acids and tetrahydrobeta acids.

55. The inorganic salt of claim 54, wherein salt comprises a monovalent or divalent cation.

56. The inorganic salt of claim 55, wherein the monovalent cation is selected from the group consisting of lithium, sodium, potassium, silver, copper.

57. The inorganic salt of claim 55, wherein the divalent cation is magnesium, calcium, barium, chromium, manganese, iron, cobalt, nickel, copper, zinc, cadmium.

58. An anti-inflammatory composition comprising the composition of any one of claim 1-11, 36 or 52 in a pharmaceutically acceptable carrier.

59. The composition of claim 58, further comprising at least one additional therapeutic agent.

60. The composition of claim 59, wherein the additional therapeutic agent is an antibiotic is selected from the group consisting of tetracyclines, doxycycline, ciprofloxacin, azitliromycin, minocycline, clarithromycin, Augmentin, penicillin, penicillin G, penicillin V, methicillin, oxacillin, carbenicillin, nafcillin, ampicillin, cephalosporins, cefaclor, cefazolin, cefuroxime, moxalactam, carbapenems, monobactams, aminoglycosides, macrolides, lincomycins, polymyxins, sulfonamides, quinolones, cloramphenical, metronidazole, spectinomycin, trimethoprim, and vancomycin.

61. A method of inhibiting a bacterial infection in a subject in need of such treatment comprising administering to the subject an effective amount of a composition of any one of claim 1-11, 34 or 46.

62. The method of claim 61, wherein the bacteria is associated with acne vulgaris.

63. The method of claim 61, wherein the bacteria is Propionibacterium acnes.

64. The method of claim 61, wherein the bacteria is Helicobacter pylori or Mycobacterium tuberculosis.

65. A method of treating acne vulgaris, the method comprising administering to a subject an effective amount of a composition of any one of claim 1-11, 36 or 52.

66. The method of claim 65, wherein the composition is administered topically.

67. The method of claim 65, wherein the composition is administered systemically.

68. A method of treating or preventing a metabolic syndrome, the method comprising administering to a subject an effective amount of a composition of any one of claim 1-11, 36 or 52.

69. The method of claim 68, wherein the composition is administered systemically.

70. The method of claim 68, wherein the method reduces the risk of heart disease or diabetes.

71. A method of treating an allergy, asthma, allergic rhinitis, or inflammation related to these conditions, the method comprising administering to a subject an effective amount of a composition of any one of claim 1-11, 36 or 52.
